(19) United States
(12) Patent Application Publication
(10) Pub. No.: US 2005/0139693 A1
(43) Pub. Date: Jun. 30, 2005

(54) Air freshening or purifying device

(75) Inventors: Paul Robinson, Hull (GB); Christopher Smith, Macclesfield (GB)

Correspondence Address: NORRIS, MCLAUGHLIN & MARCUS, 875 THIRD AVE, 18TH FLOOR, NEW YORK, NY, 10022, US

(73) Assignee: Reckitt Benckiser (UK) Limited, Slough, Berkshire (GB)

(21) Appl. No.: 10/509,541

(22) PCT Filed: Apr. 2, 2003
(86) PCT No.: PCT/GB03/01444

(30) Foreign Application Priority Data
Apr. 2, 2002 (GB) 0207516.6

(51) Int. Cl. A61L 9/04

(52) U.S. Cl. 239/60

(57) ABSTRACT

The invention relates to improvements in or relating to containers and in particular to an air freshening or purifying device utilizing a gel fragrance or a gel composition. The air freshening or purifying device comprising a container which is thermoformed from a plastic material, the container having an open side defining a gel receiving surface. The surface has therein a plurality of projections defining recesses therebetween for retaining a gel. The device further comprises a base located at a lower end of the container for supporting the container such that the gel receiving surface is in a substantially vertical orientation.

The invention relates to improvements in or relating to containers and in particular to an air freshening or purifying device utilizing a gel fragrance or a gel composition.

Many complex air freshening or purifying devices exist which use active or passive dispensation of a fragrance or air purifying composition or a combination of both. GB-A-2354711 describes a dual function dispenser, which incorporates an aerosol, which provides active dispensation, and a passive dispenser in the form of a gel cartridge, which is supported by the aerosol. However, the assembly and replacement of the two dispensers is complex. In addition an impermeable cover is required over a permeable membrane through which the fragrance can be dispersed, when the impermeable cover is removed.

U.S. Pat. No. 5,780,527 describes a gel, which can be used as a fragrancing component in an air freshening device. This gel is particularly advantageous in that it can be used in attractively shaped open containers without the need for sealing. One air freshening device, which is currently on the market, comprises an attractive glass open sided container, which is recessed to form a dish with a base and circumferential sidewall. The dish stands upright on a flattened section of its perimeter. A plurality of ridges is provided on the inner surface of the container base defining channels between the ridges, in which the gel is retained. As the fragrance is dissipated over time, the gel shrinks and cracks and is no longer wholly supported by the ridge walls. To prevent the shrinking gel from falling out of the container, a number of channels are used, which are fairly narrow or have narrow sections.

It is an object of the present invention to provide an alternative open sided air freshening or purifying device, which is cheap to manufacture and is lightweight.

According to the invention there is therefore provided an air freshening or purifying device comprising a container which is thermoformed from a plastic material, the container having an open side defining a gel receiving surface, which surface has therein a plurality of projections defining recesses therebetween for retaining a gel, said device further comprising a base located at a lower end of the container for supporting the container such that the gel receiving surface is in a substantially vertical orientation.

The use of a thermoformable plastic ensures that the device is lightweight and simple and cheap to manufacture. The form of the recesses used for gel retention means that no additional covering is necessary for retaining the gel within the container when it is supported in its vertical orientation. This simplifies the manufacture and use of the device and reduces the cost. The vertical orientation means that the gel, which may be presented in an aesthetic manner, can be easily seen. Furthermore, it lessens the amount of airborne dust, which commonly settles on horizontally supported devices.

Preferably the supporting means comprise a separate base having a slot for receiving at least a portion of the perimeter of the container.

Alternatively the supporting means may comprise a base integrally formed with the container and extending from the gel receiving surface.

This is particularly advantageous in that the device can be formed in a single thermoforming operation, which can then be filled and sealed using known production line technology.

The container is preferably formed with a circumferential rim and the rim preferably has a width greater than the depth of the gel receiving surface.

A removable cover is preferably applied to the container to cover the gel receiving surface. The removable cover is preferably a foil material which is heat sealed to the container. Alternatively a plastic laminate could be used for the removable cover.

The container is preferably filled with a gel composition, which may be a fragrance, an air purifying composition or an insecticide.

The gel is preferably a gel as described in U.S. Pat. No. 5,780,527.

A preferred embodiment of the present invention will now be described, by way of example only, with reference to the accompanying drawings in which:—

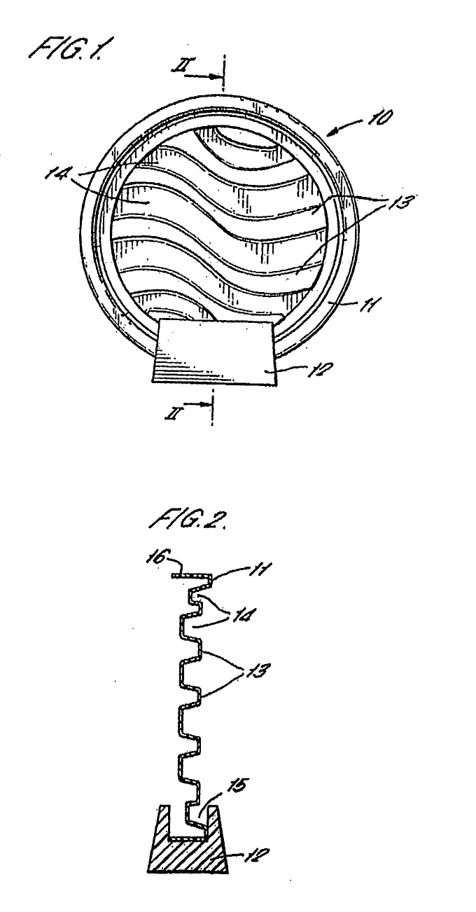

FIG. 1 is a front elevation of an air freshening device according to the present invention;

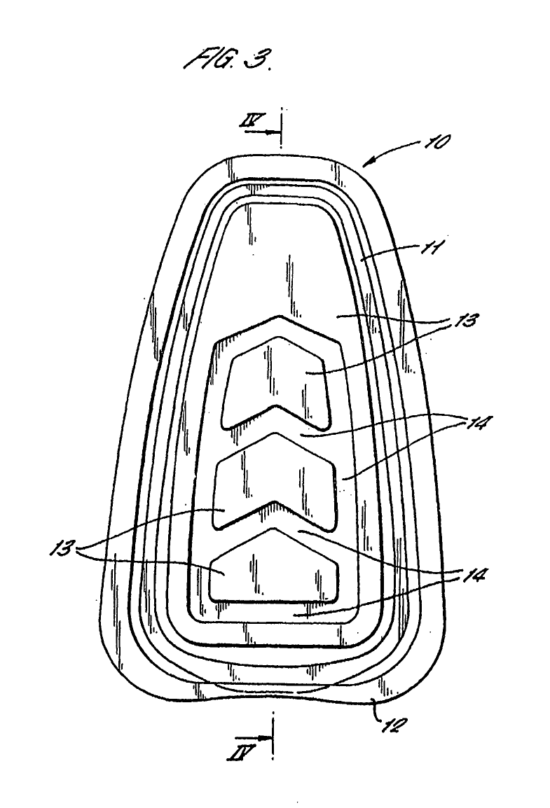

FIG. 2 is a cross-sectional side elevation of the air freshening device of FIG. 1 on the line II-II;

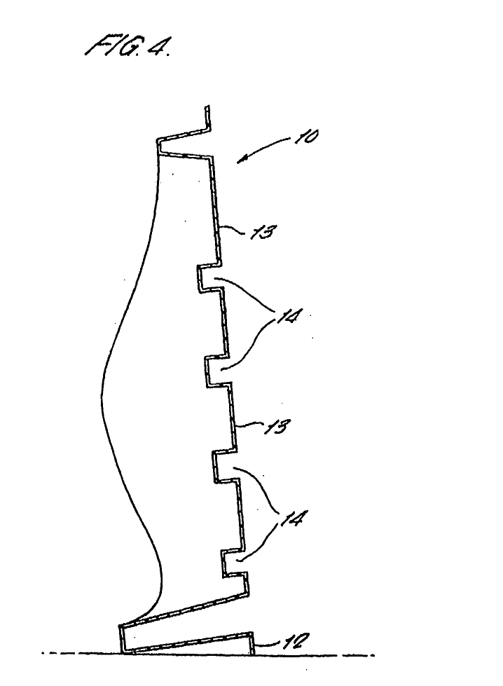

FIG. 3 is a front elevation of an alternate embodiment of the device of FIGS. 1 and 2; and

FIG. 4 is a cross sectional side elevation of the device of FIG. 3 on the line IV-IV.

Referring to FIG. 1, there is shown an air freshening (or purifying) device 10. The device 10 comprises a container 11 and a supporting base 12, located at the base of the container. The container 11 illustrated is substantially circular, although various other shapes can be used. The container 11 is preferably made from a clear, translucent and/or colored thermoformable plastic material, which is at least partially rigid and liquid impermeable.

The container 11 has a gel receiving surface which is preferably provided with a series of projections in the form of ridges 13, defining therebetween recesses in the form of channels 14. The profiles of the ridges 13 and channels 14 are not limited to that illustrated. The container 11 may have a single recess for receiving gel in an otherwise planar surface, although a series of ridges 13 and channels 14 is preferred.

It is preferred, however, that some or part of the channels 14 are reasonably narrow to hold the gel composition with which it will be filled as it shrinks or cracks over time, especially as the container 11 is intended for use with the gel receiving surface in a substantially vertical position. It may be that additional means are provided within the channels 14 or recess to assist holding the gel therein. The container 11 may have a circumferential rim 16, the depth of which is greater that the depth of the gel receiving surface. This may assist in stabilising the container 11 in the base 12.

The gel receiving surface preferably provides an attractive pattern. Thus when the container 11 is filled with a gel composition, preferably of the type described in U.S. Pat. No. 5,780,527, which is preferably strongly colored, the shape of the channels 14 is highlighted to give an attractive appearance. The gel preferably results from the cross-linking, in situ, of a homopolymer or co-polymer in the presence of a perfuming, deodorizing or insecticidal base. A suitable co-polymer is maleinised polybutadiene or polyisoprene such as Lithene N4-9000 10MA (Registered Trade Mark) obtainable from Revertex Limited. A suitable cross-linking agent, for example, is a diamine sold under the name Jeffamine 400 (Registered Trade Mark) obtainable from Huntsman Corp.

Suitable materials for the container 11 are APET, PETG, polypropylene or polyacrylonitrile as these have a high degree of clarity, are easy to thermoform and are resistant to attack by perfume. Further materials may comprise polyethylene and nylon, although these tend to be translucent or of a milky appearance or PVC, polystyrene and styreneacrylonitrile, although these may be susceptible to fragrance attack.

The supporting base 12 can be of any suitable shape, such as being substantially wedge-shaped in cross-section and is provided with a slot 15 for receiving a portion of the container 11. The base 12 and slot 15 must be dimensioned appropriate to provide sufficient support for the container 11.

Alternatively, as shown in FIGS. 3 and 4 the container 11 can be formed unitarily with the supporting base 12, such that the container 11 incorporates a supporting base section which is shaped to provide support for the overall device 10. The supporting base may be shaped to have a section which extends rearwardly from the gel receiving surface with to provide a stable support. This also means that the thermoforming tool for the combined base and container and the processing requirements for the formation step are not complicated.

The device 10 according to the present invention is particularly advantageous in that its manufacture is fairly straightforward; the devices 10 are formed in a single step thermoforming process, then filled and sealed. All of these operations can be carried consecutively using known technology on a single automated production line.

The container 11 may conveniently be provided with a removable lid covering the gel receiving surface, in the form of a tear off plastic or foil cover, to protect the gel before use or prevent it from escaping. The cover may be transparent to show the gel, or opaque or translucent. In the latter cases the cover may be printed, for example with instructions or trade marks or other information.

The air freshening or purifying device according to the present invention is convenient not only because of its lightweight nature and ease of manufacture, but the nature of the materials from which it is made means that additional packaging materials are not necessary when the devices 10 are sold.

1. An air freshening or purifying device comprising a container, the container having an open side defining a gel receiving surface, which surface has therein a plurality of projections defining recesses therebetween for retaining a gel, said device further comprising a base located at a lower end of the container for supporting the container such that the gel receiving surface is in a substantially vertical orientation, wherein the container and base are both thermoformed from a plastic material.

2. An air freshening device or purifying device according to claim 1 in which the base is formed with a slot for receiving at least a portion of the perimeter of the container.

3. An air freshening or purifying device according to claim 1 in which the base is integrally formed with the container.

4. An air freshening or purifying device comprising a container, the container having an open side defining a gel receiving surface, which surface has therein a plurality of projections defining recesses therebetween for retaining a gel, said device further comprising a base formed with a slot for receiving at least a portion of a lower end of the perimeter of the container for supporting the container such that the gel receiving surface is in a substantially vertical orientation, wherein the container and base are both thermoformed from a plastic material.

5. An air freshening or purifying device comprising a container, the container having an open side defining a gel receiving surface, which surface has therein a plurality of projections defining recesses therebetween for retaining a gel, said device further comprising a base located at and integrally formed with a lower end of the container for supporting the container such that the gel receiving surface is in a substantially vertical orientation, wherein the container and base are both thermoformed from a plastic material.

6. An air freshening or purifying device according to claim 1 in which the container is formed with a circumferential rim.

7. An air freshening or purifying device according to claim 6 in which the rim has a width greater than the depth of the gel receiving surface.

8. An air freshening or purifying device according to claim 1 further comprising a removable cover applied to the container to cover the gel receiving surface.

9. An air freshening or purifying device according to claim 8 in which the removable cover is made from a foil material which is heat sealed to the container.

10. An air freshening or purifying device according to claim 8 in which the removable cover is made from a plastic laminate which is heat sealed to the container.

11. An air freshening or purifying device according to claim 1 in which the container is filled with a gel composition.

12. An air freshening or purifying device according to claim 10 in which the gel composition is a fragrance.

13. An air freshening or purifying device according to claim 10 in which the gel composition is an insecticide.

14. An air freshening or purifying device according to claim 10 in which the gel composition is an air purifying composition.

15. An air freshening or purifying device according to claim 4 in which the container is formed with a circumferential rim.

16. An air freshening or purifying device according to claim 5 in which the container is formed with a circumferential rim.

17. An air freshening or purifying device according to claim 1 further comprising a removable cover applied to the container to cover the gel receiving surface.

18. An air freshening or purifying device according to claim 4 further comprising a removable cover applied to the container to cover the gel receiving surface.

19. An air freshening or purifying device according to claim 5 further comprising a removable cover applied to the container to cover the gel receiving surface.
